(19) United States
(12) Patent Application Publication
(10) Pub. No.: US 2011/0183389 A1
(43) Pub. Date: Jul. 28, 2011

(54) PRODUCTION OF LACTIC ACID FROM HEMICELLULOSE EXTRACTS

(76) Inventors: G. Peter van Walsum, Orono, ME (US); Sara L. Walton, Glenburn, ME (US); Adriaan Reinhard Pieter van Heiningen, Orono, ME (US)

(21) Appl. No.: 12/912,283

(22) Filed: Oct. 26, 2010

Related U.S. Application Data
(60) Provisional application No. 61/254,772, filed on Oct. 26, 2009.

(51) Int. Cl. C12P 7/56

(52) U.S. Cl. 435/139

(57) ABSTRACT

A method is provided for producing lactic acid comprising fermenting sugars derived from biomass using sugar consuming bacteria to produce lactic acid. In certain embodiments, the biomass is woody biomass, and the bacteria are pentose consuming bacteria such as Bacillus coagulans.

### CROSS-REFERENCE TO RELATED APPLICATIONS

This application claims the benefit of U.S. Provisional Application No. 61/254,772, filed Oct. 26, 2009, the disclosure of which is incorporated herein by reference.

### BACKGROUND OF THE INVENTION

This invention relates in general to the production of useful products from biomass, and in particular to the production of lactic acid from hemicellulose extracts obtained from biomass.

Lactic acid has a variety of uses in the food and pharmaceutical industries, and was identified by the USDOE as one of the top 30 potential building block chemicals from biomass. Lactic acid has the potential to replace chemicals currently derived from petrochemical routes, such as acrylic acid, or the ability to form novel bio-products such as polylactic acid. Lactic acid may be produced by synthetic or fermentation routes. Synthetic production uses lactonitrile as a starting material and produces a racemic mixture. Fermentation processes have become more common because they produce either D- or L-lactic acid at chiral purity near 100%. Both isomers can be polymerized but the properties of the polymer vary with the stereo-purity. Optically pure lactic acid is important to the formation of polymers with desirable mechanical properties.

In view of the many beneficial uses of lactic acid, and the availability of large quantities of biomass, it would be desirable to provide a method for the production of lactic acid from biomass.

### SUMMARY OF THE INVENTION

A method is provided for producing lactic acid comprising fermenting sugars derived from biomass using sugar consuming bacteria to produce lactic acid. In certain embodiments, the biomass is woody biomass, and the bacteria are pentose consuming bacteria such as Bacillus coagulans.

### BRIEF DESCRIPTION OF THE DRAWINGS

FIG. 1 includes graphs showing the effect of acetic acid concentration on xylose consumption.

FIG. 2 includes graphs showing the effect of sodium concentration on xylose consumption.

FIG. 3 includes graphs relating to fermentation of a five sugar model representative of larch extract.

FIG. 4 includes graphs relating to fermentation of a hot water extracted larch.

FIG. 5 is a graph relating to fermentation of northern hardwood green liquor extract.

FIG. 6 is a graph relating to fermentation of hot water extracted southern hardwood.

FIG. 7 is a graph showing a comparison of lactic acid and ethanol fermentations of green liquor extracts.

### DETAILED DESCRIPTION OF THE PREFERRED EMBODIMENT

A method is provided for producing lactic acid. The method comprises fermenting sugars derived from biomass using sugar consuming bacteria to produce the lactic acid. Any suitable biomass can be used in the method. In certain embodiments, the biomass is a cellulose containing biomass, and more particularly the biomass can be a woody biomass.

The sugars can be any suitable sugars derived from biomass. In certain embodiments, when the biomass is a woody biomass, the sugars include glucose, mannose, galactose, xylose and arabinose. Also, in certain embodiments, the sugars are hemicellulose sugars. The hemicellulose sugars can be provided from any suitable source. In certain embodiments, the hemicellulose sugars are provided by aqueous extraction of wood. This can include any extraction with any suitable aqueous material. In certain embodiments, the aqueous material is water, green liquor, or a mixture of water and green liquor.

The sugars can be fermented using any suitable sugar consuming bacteria. In certain embodiments, the bacteria are pentose consuming bacteria, examples of which include Bacillus coagulans, Bacillus smithil, and Lactobacillus vini. In a particular embodiment, the bacteria is Bacillus coagulans of any pentose fermenting strain, for example, Bacillus coagulans MXL-9, B. coagulans NRS-185, or B. coagulans B-14317.

In certain particular embodiments, the method relates to the production of lactic acid from hemicellulose extracts. As described in more detail below, Bacillus coagulans MXL-9 was found capable of growing on pre-pulping hemicellulose extracts, utilizing all of the principle monosugars found in woody biomass. This organism is a moderate thermophile isolated from compost for its pentose utilizing capabilities. It was found to have high tolerance for inhibitors such as acetic acid and sodium which are present in pre-pulping hemicellulose extracts.

Fermentation of 20 g/L xylose in the presence of 30 g/L acetic acid required a longer lag phase but overall lactic acid yield was not diminished. Similarly fermentation of xylose in the presence of 20 g/L sodium increased the lag time but did not affect overall product yield, though 30 g/L sodium proved completely inhibitory. Fermentation of hot water extracted Siberian larch containing 45 g/L total monosaccharides, mainly galactose and arabinose, produced 33 g/L lactic acid in 60 hrs and completely consumed all sugars. Small amounts of co-products were formed, including acetic acid, formic acid and ethanol.

Hemicellulose extract formed during autohydrolysis of mixed hardwoods contained mainly xylose and was converted into lactic acid with a 94% yield. Green liquor extracted hardwood hemicellulose containing 10 g/L acetic acid and 6 g/L sodium was also completely converted into lactic acid at a 72% yield. The Bacillus coagulans MXL-9 strain was found to be well suited to production of lactic acid from lignocellulosic biomass due to its compatibility with conditions favorable to industrial enzymes and its ability to withstand inhibitors while rapidly consuming all pentose and hexose sugars of interest at high product yields.

Bacillus coagulans is a spore-forming thermophilic lactic acid bacteria first isolated from spoiled milk and tomato juice. Strain MXL-9 was isolated by the USDA ARS from dairy manure compost for its ability to consume pentose sugars. It is a moderate thermophile, growing at 50-55° C. and producing mainly L-lactic acid. The ability of Bacillus coagulans to utilize a wide range of sugars under thermophilic conditions makes it well suited to the conversion of lignocellulosic biomass.

One promising development in conversion of lignocellulosic biomass to renewable fuels and chemicals is the process of pre-pulping hemicellulose extraction. Extracting hemicellulose prior to pulping creates a new feedstock within the existing pulp and paper industry while preserving cellulose for production of the more valuable pulp. In present-day kraft pulp mills, hemicellulose is burned during chemical recovery along with lignin to generate power and steam. Because hemicellulose does not have a high heating value, conversion by biological fermentation processes offers a potential way to increase the value derived from lignocellulosic feedstocks within an integrated bio-refinery.

Hemicellulose extraction can be achieved by autohydrolysis in the presence of water prior to the manufacture of dissolving pulp grades, or alternatively in the presence of alkaline chemicals which are necessary to maintain pulp yields in the manufacture of kraft pulp. Extraction of hardwood species generates an extract rich in xylan oligosaccharides and acetic acid. Softwood extracts have lower acetic acid and are higher in galactose, mannose and arabinose. All extracted solutions contain low concentrations of glucose derived from dissolution of the amorphous low molecular weight cellulose and from glucomannans. The major portion of cellulose is preserved for pulp production because it achieves greater value as fiber than as a feedstock for commodity fuels and chemicals.

In certain embodiments of the present method, substantially all the sugars derived from woody biomass, glucose, mannose, galactose, xylose and arabinose, are utilized by the fermentation organism with high conversion yields to the desired product. In certain embodiments, the concentration of product achieved in the fermentation broth is high to overcome the costs of recovery and purification.

### Experimental Work

The following work details a study on the fermentation of hemicellulose derived through extraction of both hardwoods and softwoods prior to pulping. Bacillus coagulans MXL-9 was tested on pure substrates to determine its ability to consume the pentose sugars xylose and arabinose. It was also tested in the presence of varying levels of acetic acid and sodium which are potential inhibitors to bacterial growth at the concentrations contained in hemicellulose extracts.

Materials and Methods

Hemicellulose Extraction

Mixed hardwood chips consisting mainly of maple and lesser amounts of beech, poplar and birch were obtained from International Paper (Jay, Me.). Woodchips contained 48% moisture (wet basis) and were not screened or dried. Woodchips were extracted with either water or 2% total titratable alkali (TTA) of green liquor in a custom-built rotating digester (Hodgins, University of Maine, Orono, Me.). Green liquor was comprised of 0.88 g/L NaOH, 2.57 g/L Na₂S, and 8.16 g/L Na₂CO₃. In each batch, 7 kg of oven-dry wood was added to the digester at a liquor to wood ratio of 4:1 L/kg, which includes the wood moisture. All cooks also contained anthraquinone (AQ) which was charged at 0.05% on a dry wood basis. AQ addition has been shown to increase pulp yield and delignification. The extraction was performed at a temperature of 160° C. and H-factor of 800 hrs at each chemical loading. The extracted woodchips underwent kraft pulping. Larch extracts were received from the Helsinki University of Technology (Finland), where they were prepared by hot water extraction of Siberian larch at 160° C. for 60 min. in a rotating autoclave.

Ultrafiltration of Hemicellulose Extracts

Ultrafiltration of 2% green liquor extracts was performed using a Kerasep ceramic membrane system (Novasep, France). The ceramic membrane is constructed of monolithic TiO₂—Al₂O₃ containing 19 channels for a total surface area of 0.08 m². The operating pressure was 50-60 psi, with a pressure drop across the membrane of 2 psi. A centrifugal pump with a maximum flow rate of 13 gal/min was used and the minimum operating volume was 4 L. The membrane cut-off size was 50 kD. The system was operated in continuous recycle mode beginning with 35 L of hemicellulose extract. Permeate was removed while retentate was returned to the feed tank until 30 L of permeate were collected, a 7-fold concentration of the hemicelluloses. The maximum temperature reached was 54° C. Additionally a 15 kD membrane was used to concentrate 14.1 L of 4% green liquor extract down to 4.3 L, a 3-fold concentration.

Hydrolysis of Extraction Liquor

Samples were hydrolyzed at pH 1.0 with sulfuric acid in an autoclave (Hirayama, Japan) at 120° C. for 60 min. After hydrolysis, the solutions were filtered through a glass microfiber filter to remove Klason lignin. The solution pH was raised to neutral by addition of solid calcium hydroxide and subsequently filtered through glass microfiber filters to remove the resulting gypsum.

Fermentation

Bacillus coagulans MXL-9 was provided by the USDA ARS National Center for Agricultural Utilization Research (Peoria, Ill.), and stock cultures maintained on media containing 10 g/L tryptone, 5 g/L yeast extract, 2 g/L K₂HPO₄ and 1.5% agar (if applicable). Fermentation was performed in 400 mL DASGIP bio-reactors with a working volume of 250 mL (DASGIP BioTools, Shrewsbury, Mass.). The pH was maintained at 6.5 by automatic addition of 2N KOH. Vessels were sparged with nitrogen prior to inoculation and maintained negative redox values, indicating anaerobic growth. Temperature was maintained at 50° C. and agitation at 250 RPM by magnetic stirring. Vessels containing growth media (tryptone, yeast extract, K₂HPO₄) and hemicellulose extract (if applicable) were autoclaved at 121° C. for 20 min. to sterilize prior to aseptic additions. Minimal salts solution containing 26.1 g/L K₂HPO₄, 11.3 g/L KH₂PO₄, and 25 g/L NH₄NO₃ was added aseptically to growth media at 20 mL/L. After autoclaving, 1 mL/L of the following sterile stocks were added: 1.05 M Nitrilotriacetic acid, 0.59 M MgSO₄.7H₂O, 0.91 M CaCl₂.2H₂O, and 0.04 M FeSO₄.7H₂O. For fermentation of pure xylose, a 100 g/L solution was autoclaved separately from growth media. Inoculating cultures were grown in the same media with 20 g/L xylose in all experiments. For inhibition experiments, acetic acid was added in the form of ammonium acetate and sodium was added in the form of sodium sulfate. The inoculum represented 5% of the working volume.

Fermentation to produce ethanol from hemicellulose extracts was performed by Escherichia coli K011. Media contained 20 g/L LB and the antibiotic chloramphenicol was added at 40 mg/L to select for only the E. coli K011 strain. Thiamine was added at 1 mg/L and a trace metals solution at 5 mL/L, consisting of per liter: 5 g disodium EDTA, 0.22 mg zinc sulfate heptahydrate, 0.5 g calcium chloride, 0.5 g ferrous sulfate, 0.1 g ammonium molybdate tetrahydrate, 0.16 g cupric chloride, 0.16 g cobalt chloride. The inoculum represented 5% of the working volume. Temperature was maintained at 37° C. and agitation at 250 RPM. The pH was controlled at 7.0 by addition of 2N KOH.

Chemical Analyses

Lactic acid, ethanol, acetic acid, xylose and furans were analyzed by high performance liquid chromatography (HPLC) equipped with refractive index and UV detection (Shimadzu, Columbia, Md.), using an Aminex HPX-87H(H) column (Bio-Rad, Hercules, Calif.). The column was operated with a 5 mM sulfuric acid mobile phase at a flow rate of 0.6 mL/min and oven temperature of 60° C. Samples were filtered through 0.22 μm syringe filters or centrifuged for 10 min. at 14,000 G prior to injection. Hemicellulose extracts were also measured using an Aminex HPX-87P(P) column with a water mobile phase at a flow rate of 0.6 mL/min and oven temperature of 80° C. to separate glucose, xylose, mannose, galactose and arabinose. Internal standards of fucose were used for the H-column and erythritol for the P-column. Klason lignin was determined gravimetrically, and acid soluble lignin was determined by TAPPI method 250.

Results and Discussion

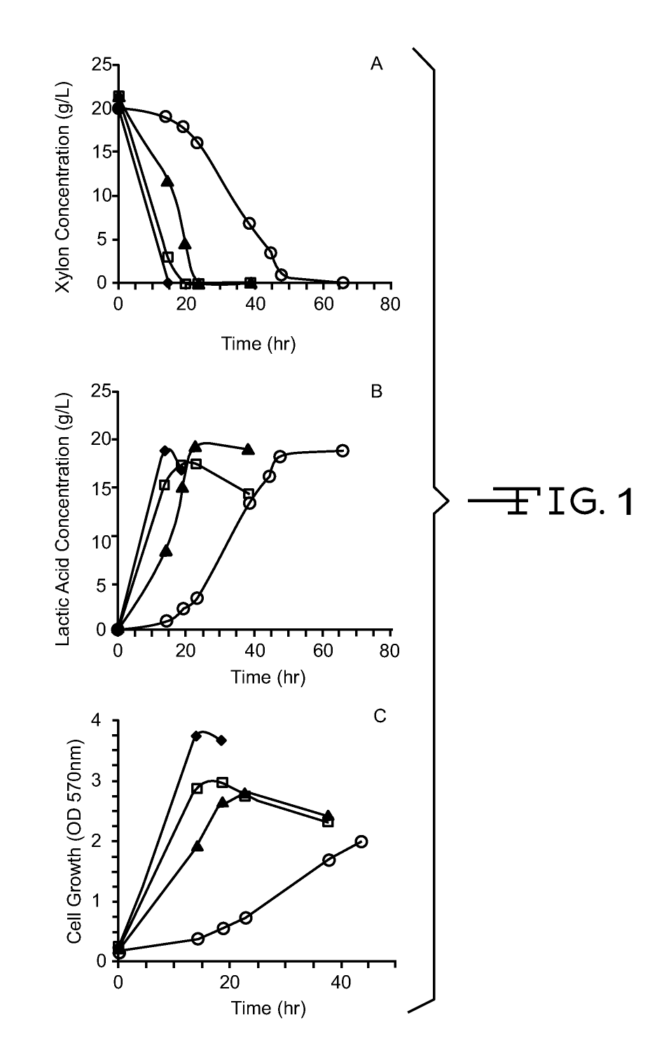

An assessment of the suitability of Bacillus coagulans MXL-9 for conversion of hemicellulose extracts to lactic acid began with testing its ability to withstand the inhibitory chemicals that have previously been shown to be detrimental to cell growth of E. coli and other microbial cultures. Acetic acid and sodium inhibition were each evaluated at concentrations ranging from 0 to 30 g/L in bio-reactors controlled at a pH of 6.5. On average, an uninhibited control containing 20 g/L of xylose produced lactic acid at 90% conversion, or 18 g/L of lactic acid. Minor additional side products included acetic acid, formic acid, and ethanol. FIG. 1 shows the effect of increased acetic acid concentration on xylose consumption, lactic acid production and cell growth. The control was able to completely consume xylose within 14 hours, while higher acetic acid levels increased the fermentation time. Fermentations at concentrations of 10 and 20 g/L acetic acid were complete in under a day, but at 30 g/L the fermentation required two days. Despite the slower growth rates, the overall product yields for lactic acid remained high and were all similar to that of the control. Green liquor hemicellulose extracts may contain as much as 10 g/L acetic acid, which could be further concentrated if evaporation methods are employed to increase the monosaccharide concentration prior to fermentation. Acetic acid can be removed before fermentation by liquid-liquid extraction if necessary, though the concentration is not high enough to warrant removal if oligosaccharides are concentrated by ultrafiltration. Hemicellulose extracts produced by water extraction do not contain acetic acid at high enough concentrations to inhibit fermentation significantly.

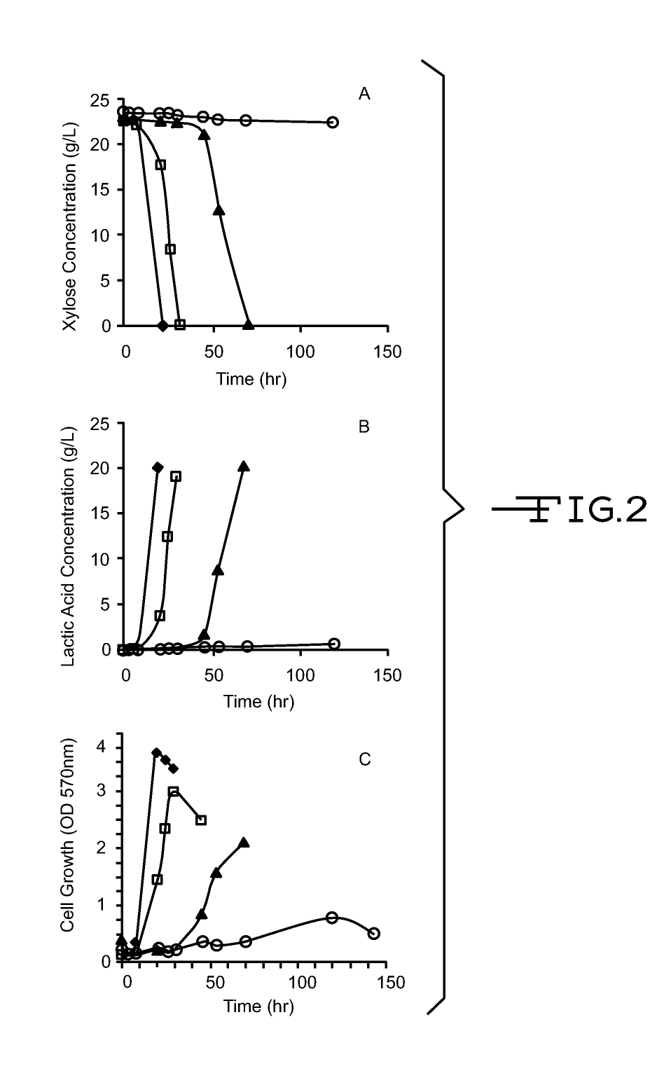

When alkaline chemicals such as green liquor are used to perform hemicellulose extraction the residual sodium concentration can also impact the level of microbial inhibition. For an extract made with 2% green liquor the sodium concentration is 3 g/L. If evaporation methods were used to concentrate the dilute monosaccharides 10-fold to 30 g/L sodium, the data in FIG. 2 suggests that no xylose consumption could be expected. For sodium concentrations up to 20 g/L the organism required a longer lag phase, but was able to adjust and completely consume xylose with yields comparable to that of the control. However, at 30 g/L sodium no cell growth occurred over the course of 6 days. Table 1 summarizes the product yields obtained at varying concentrations of acetic acid and sodium. The tolerance of Bacillus coagulans MXL-9 to these inhibitors was found to be sufficiently high to predict that it could grow in hemicellulose extracts.

The hemicellulose extracts that were tested for fermentation by Bacillus coagulans MXL-9 include hot water extracted Siberian larch (softwood), hot water extracted mixed hardwoods, and ultra-filtered 2% and 4% green liquor extracted hardwood. Extracts were hydrolyzed by sulfuric acid prior to fermentation and then neutralized. Compositional analysis of the hemicellulose extracts tested both before and after fermentation is given in Table 2. Hot water extracted larch contained the highest concentration of monosaccharides, which were mainly derived from arabinogalactans. Galactose comprised 55.1% of the available sugar, while arabinose represented 15.6%. Xylose (12.5%), mannose (11.7%) and glucose (5.1%) were also present, resulting in a total of 45 g/L monosugars available for fermentation. Extracts produced from the mixed hardwood chips contained xylose as the principle sugar. Hydrolyzed hot water extracts contained 21.4 g/L of sugar, comprised of 70% xylose, and 7-8% each of galactose, mannose, glucose and arabinose. Hot water extracts do not have the issue of sodium inhibition, and acetic acid is present at lower concentrations than in alkaline extracts, at 1.9 g/L in larch extracts and 5.8 g/L in hardwood extracts. Furfural concentration is slightly higher in the hot water extracts compared to alkaline, as is acid soluble lignin, both of which are potential inhibitors of cell growth.

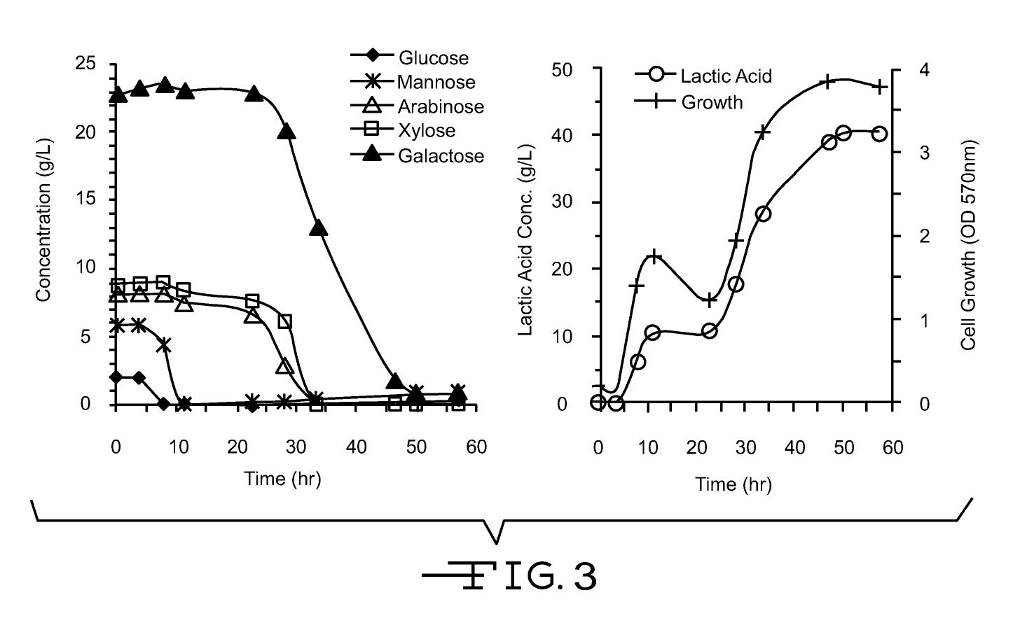

Bacillus coagulans MXL-9 is capable of consuming all five of the monosaccharides found in lignocellulose. The organism has a marked preference for glucose and mannose, where FIG. 3 shows that both of these hexose sugars in a model sugar system representative of larch extracts were consumed rapidly in the first 10 hours of fermentation. After depletion of glucose and mannose there was an extended 12 hour lag phase where the metabolism shifted, after which consumption of arabinose, xylose and galactose began. After 50 hours the organism had produced 40.5 g/L of lactic acid by consuming 45.8 g/L of monosaccharides, for a yield of 88.6%. Cell growth, as measured by optical density, is shown for the model sugar systems but is not available for hemicellulose extracts which have too many suspended solids that result in optical interference.

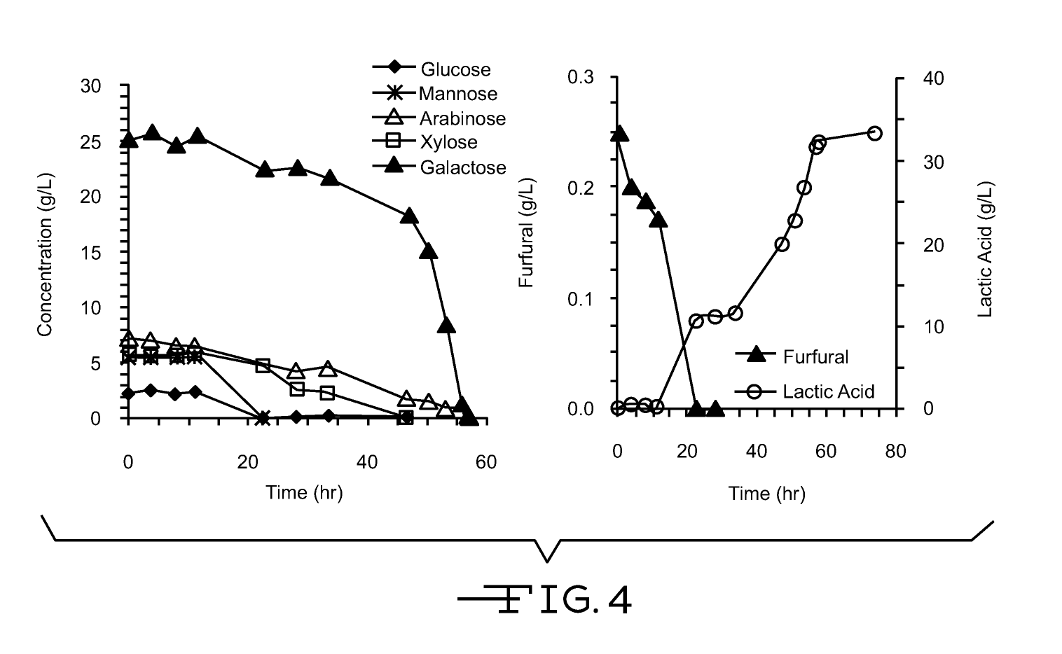

The actual larch extract contained several inhibitory substances such as acetic acid, furfural, HMF and lignin degraded phenolics which resulted in a longer initial lag phase and lower yield. Again glucose and mannose were consumed first, followed by a 12 hour lag before xylose, arabinose and galactose consumption. FIG. 4 shows that fermentation was complete after 58 hours, where 33 g/L lactic acid was produced from 44 g/L sugar, at a yield of 75%. Furfural (0.25 g/L) was entirely metabolized by the organism within 20 hours, before metabolism of xylose, arabinose and galactose commenced. A variety of other bacteria and yeasts have been shown to possess enzymes for transforming furfural into either furfuryl alcohol or furoic acid which are less toxic to the cells. Organisms with the ability to metabolize furfural have been investigated as potential biological detoxification agents in the treatment of lignocellulosic biomass. Bacillus coagulans MXL-9 is a promising organism for fermentation of lignocellulose feedstocks due to its ability to simultaneously detoxify the inhibitory furans while converting all of the sugars into lactic acid at high yields.

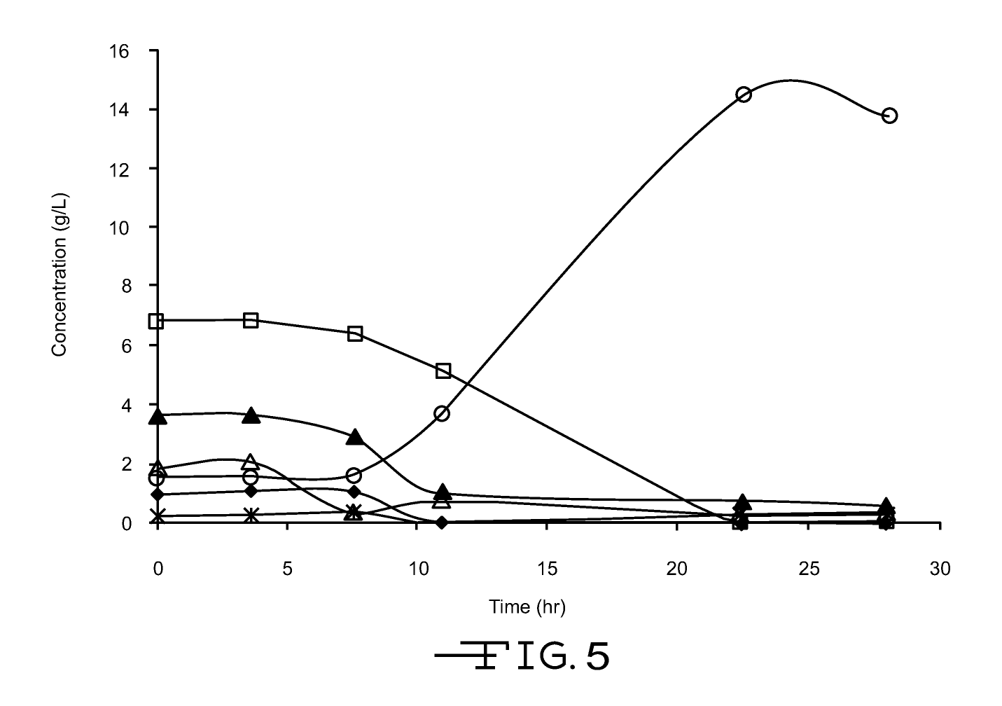

The 4% green liquor extract shown in FIG. 5 had been concentrated three-fold by ultrafiltration at 15 kilodaltons. The total monosaccharide concentration before fermentation was 15.6 g/L and contained mainly xylose as shown in Table 2. Fermentation was complete after 22 hours. For hardwood hemicellulose extracts an intermediate lag phase was not required for metabolism to shift, likely due to the much lower concentrations of furfural, galactose and arabinose. Green liquor extracts initially contained 1.5 g/L of lactic acid formed from sugar degradation through the alkaline peeling reactions, and an additional 13 g/L of lactic acid were produced by the organism during fermentation. The yield on lactic acid produced through fermentation was 83%. The green liquor extracts contain the highest acetic acid concentration, 8.9 g/L. While acetic acid is sometimes a byproduct of Bacillus coagulans, no additional acetic acid was formed, nor were any other byproducts.

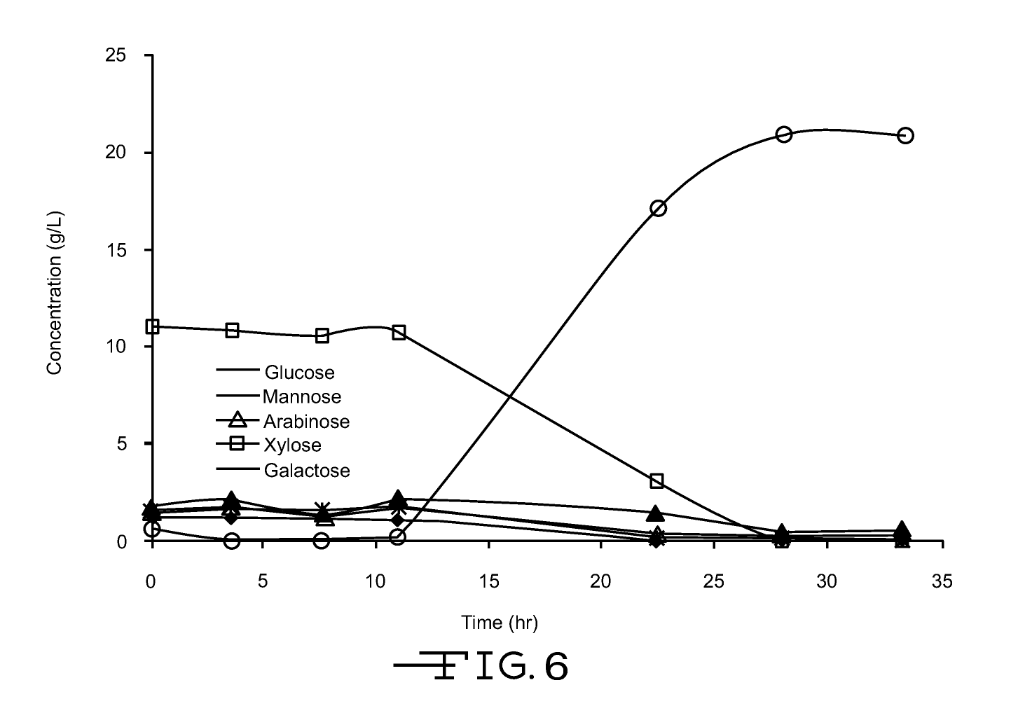

Fermentation of hot water extracted mixed southern hardwoods is shown in FIG. 6. The extracts contained 21.4 g/L of monosaccharides, primarily xylose, and 5.8 g/L acetic acid. Following an initial lag phase of 10 hours, all of the sugars were consumed within 24 hours without an intermediate lag phase. Production of 20.8 g/L lactic acid represented a 94% yield based on the initially available monosaccharides, excluding the lactic acid already present in the extracts. Table 2 shows that no byproducts of acetic acid or ethanol were formed and formic acid showed only a minor increase. Furfural (0.4 g/L) was completely metabolized by the organism.

Bacillus coagulans is well suited to simultaneous saccharification and fermentation (SSF) because of its compatibility with the temperature and pH optima of enzymes. In addition some strains of this organism have inherent hemicellulose degrading enzymes, such as strain BL69 which contains xylanase activity. Fermentation of unhydrolyzed hemicellulose extracts directly into lactic acid without the need for additional enzyme or chemical hydrolyzing agents would be the ideal performance. Experiments with strain MXL-9 showed that unhydrolyzed hot water extracts of hardwood contained 5 g/L of monosugars and an estimated 19 g/L of oligomeric sugars at the start of fermentation. After a week of fermentation, the culture produced 5 g/L of lactic acid, which indicates that there was not a significant breakdown of oligomeric sugars, but all sugar present in monomeric form was utilized.

Fermentation of hemicellulose extracts into lactic acid has an advantage over the fermentative production of fuel ethanol because the metabolic pathway does not result in production of carbon dioxide. Production of ethanol has a maximum yield of only 0.51 grams of ethanol per gram of product due to CO₂ formation, whereas lactic acid producing organisms do not cycle as much carbon into waste products and can therefore achieve higher product yield. This yield increase is particularly beneficial in a process such as converting hemicellulose extracts, which are relatively low in sugar content and high in inhibitor content. Increasing the sugar concentration prior to fermentation is necessary to achieve a high enough product titer for an economically viable process. Methods for increasing sugar concentration also increase the levels of inhibitors such as lignin, and may increase organic acid and salt concentrations. If the fermentation yield can be doubled by avoiding CO₂ generation then only half as much effort is expended to concentrate the feedstock and inhibitors only accumulate by half. Lactic acid is therefore easier than fuel ethanol to produce in an economically feasible manner. The production scale of a typical forest products mill is also better suited to high-value, low volume products than to lower value commodities.

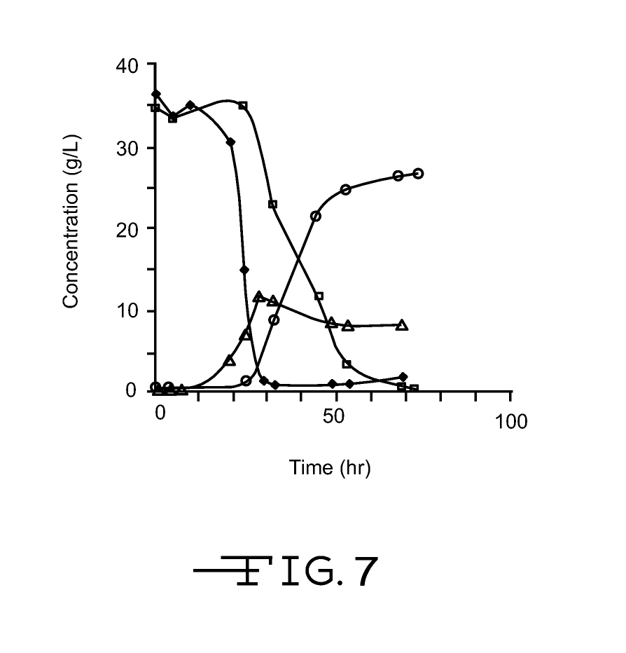

A direct comparison of ethanol and lactic acid fermentation is shown in FIG. 7. The feedstock was a 2% green liquor extract of hardwood chips which had been concentrated 7-fold by ultrafiltration at 50 kD. The initial monosaccharide concentration was 35 g/L and consisted predominantly of xylose with 4-7% each of glucose, arabinose, mannose and galactose. Also present initially were 12 g/L of acetic acid, 3 g/L sodium, 1.8 g/L of formic acid and 0.7 g/L of lactic acid. Fermentation into ethanol by E. coli K011 was slightly faster but far less efficient than the fermentation into lactic acid by B. coagulans MXL-9. The highest ethanol titer of 12 g/L was obtained after just 28 hrs but represents a yield of only 0.32 g/g based on the initial sugar concentration, or 63% of maximum theoretical ethanol production. Alternately the highest lactic acid titer was 26 g/L obtained at 68 hrs, and neglecting the lactic acid initially present in the extract itself, this represents at 74% production yield. Comparing the products on the basis of each organism's theoretical maximum, B. coagulans was 1.18 times more efficient than E. coli. Lactic acid was shown to be produced at more than twice the concentration of ethanol from the concentrated green liquor hemicellulose extracts.

### CONCLUSIONS

Bacillus coagulans MXL-9 is capable of consuming all of the principle monosaccharides found in hemicellulose extract and producing lactic acid at high yields. In softwood extracts glucose and mannose are consumed preferentially, followed by an intermediate lag phase during which metabolism shifts to xylose, arabinose and galactose consumption. In hardwood extracts the same preference for glucose and mannose was observed but an intermediate lag phase was not required. This organism has relatively high tolerance for inhibitors found in hemicellulose extract including acetic acid and sodium, and has the ability to detoxify furfural. The ability to consume a wide range of sugars, grow at 50° C. and pH 5-7 makes this bacteria well suited to SSF operations for lignocellulosic feedstocks. Hemicellulose extracts containing 45 g/L of monosaccharides were converted into 33 g/L lactic acid, which represents a 14% decrease in yield and 8 hours increased fermentation time compared to fermentation of monosaccharides in defined media containing the same amounts of each sugar.

TABLE 1
Product yields achieved by Bacillus coagulans MXL-9 in the presence of varying inhibitor concentrations.

| | | | | Formic | Acetic | | | | Total |
|---|---|---|---|---|---|---|---|---|---|
| Inhibitor | Ferment | Xylose | Lactic Acid | Acid | Acid | Ethanol | Lactic Acid | Lactic Acid | Products |
| Conc. | Time | Consumed | Produced | Produced | Produced | Produced | Prod. Rate | Yield | Yield |
| (g/L) | (hr) | (g/L) | (g/L) | (g/L) | (g/L) | (g/L) | (g/L/hr) | (%) | (%) |
| 0 HAc | 14 | 20.1 | 18.8 | 1.4 | 0.7 | 1.2 | 1.3 | 93.2 | 109.5 |
| 10 HAc | 17 | 21.4 | 17.3 | 1.1 | 0.0 | 1.0 | 1.0 | 81.0 | 90.7 |
| 20 HAc | 22 | 20.6 | 19.3 | 0.5 | 0.0 | 0.8 | 0.9 | 93.6 | 99.8 |
| 30 HAc | 60 | 20.2 | 18.5 | 0.4 | 0.0 | 0.4 | 0.3 | 91.4 | 95.3 |
| 0 Na | 19 | 22.5 | 19.8 | 1.7 | 0.9 | 0.1 | 1.0 | 87.3 | 99.3 |
| 10 Na | 32 | 22.4 | 18.8 | 1.3 | 0.8 | 0.0 | 0.6 | 83.1 | 92.4 |
| 20 Na | 62 | 22.7 | 19.6 | 1.3 | 0.7 | 0.0 | 0.3 | 85.9 | 94.5 |
| 30 Na | 150 | 0.8 | 0.0 | 0.0 | 0.0 | 0.0 | 0.0 | 2.6 | 2.6 |

TABLE 2
Composition of hemicellulose extracts before and after fermentation by
Bacillus coagulans MXL-9

| | Concentration (g/L) | | | | | |
|---|---|---|---|---|---|---|
| | Hot Water | | Conc. Green | | Hot Water | |
| | Extracted | | Liquor Extracted | | Extracted Siberian | |
| | Hardwood | | Hardwood | | Larch | |
| | 0 hrs | 28 hrs | 0 hrs | 22 hrs | 0 hrs | 73 hrs |
| Glucose | 1.6 | 0.0 | 1.0 | 0.0 | 2.3 | 0.0 |
| Mannose | 1.6 | 0.2 | 0.2 | 0.0 | 5.4 | 0.0 |
| Galactose | 1.7 | 0.5 | 3.7 | 0.7 | 25.3 | 0.0 |
| Xylose | 14.9 | 0.0 | 8.9 | 0.0 | 5.7 | 0.0 |
| Arabinose | 1.5 | 0.0 | 1.8 | 0.0 | 7.1 | 0.0 |
| Lactic Acid | 0.7 | 20.9 | 1.6 | 14.5 | 0.1 | 33.5 |
| Formic Acid | 0.9 | 1.3 | 2.4 | 2.3 | 0.5 | 2.5 |
| Acetic Acid | 5.8 | 5.8 | 8.9 | 8.8 | 1.9 | 3.3 |
| Ethanol | 0.0 | 0.0 | 0.0 | 0.0 | 0.0 | 0.0 |
| Sodium | 0.0 | 0.0 | 3.0 | 0.0 | 0.0 | 0.0 |
| Furfural | 0.4 | 0.0 | 0.0 | 0.0 | 0.3 | 0.0 |
| Total sugar | 21.4 | 0.7 | 15.6 | 0.7 | 45.9 | 0.0 |

What is claimed is:

1. A method for producing lactic acid comprising fermenting sugars derived from biomass using sugar consuming bacteria to produce lactic acid.

2. The method of claim 1 wherein the sugar consuming bacteria are pentose consuming bacteria.

3. The method of claim 2 wherein the pentose consuming bacteria are Bacillus coagulans.

4. The method of claim 3 wherein the pentose consuming bacteria are Bacillus coagulans MXL-9.

5. The method of claim 1 wherein the biomass is woody biomass.

6. The method of claim 5 wherein the sugars include glucose, mannose, galactose, xylose and arabinose.

7. The method of claim 5 wherein the sugars are hemicellulose sugars.

8. The method of claim 1 further comprising aqueous extraction of wood to produce the hemicellulose sugars to be fermented.

9. The method of claim 8 wherein the aqueous extract is water, green liquor, or a mixture of water and green liquor.

10. The method of claim 1 which includes simultaneous saccharification and fermentation of the sugars.

11. The method of claim 8 which includes concentration of the extract by ultrafiltration or evaporation.

12. The method of claim 8 which includes hydrolysis of the extract by acid or enzyme-catalyzed hydrolysis.

13. The method of claim 8 which includes removal of acetic acid from the extract prior to fermentation.

14. The method of claim 13 which includes liquid-liquid extraction to remove acetic acid from the extract.
